(19) United States
(12) Patent Application Publication
(10) Pub. No.: US 2026/0137096 A1
(43) Pub. Date: May 21, 2026

(54) TEA AND PLANT CELL CULTURE GROWN IN VITRO COMPOSITION

(71) Applicant: BIO HARVEST LTD., Rehovot (IL)

(72) Inventors: Yoheved HAGAY, Rehovot (IL); Malkit AZACHI, Rehovot (IL)

(21) Appl. No.: 19/370,638

(22) Filed: Oct. 27, 2025

Related U.S. Application Data
(60) Provisional application No. 63/721,588, filed on Nov. 18, 2024.

(51) Int. Cl. A23F 3/14; C12N 5/04

(52) U.S. Cl. CPC A23F 3/14; C12N 5/04

(57) ABSTRACT

The invention relates to a composition for making a tea beverage, the composition comprising tea which may be ground tea in combination with plant cell culture grown in vitro in a large scale. The plant cell culture grown in vitro in a large scale may be derived from any plant, such as a fruit including but not limited to grape berry, pomegranate, olive, and the like. The composition may be inserted into a pod and be used in a machine or inserted into a tea bag or a container.

### CROSS REFERENCE TO RELATED APPLICATIONS

This application claims the benefit of priority of U.S. Provisional Patent Application No. 63/721,588, filed Nov. 18, 2024, the contents of which are incorporated herein by reference in their entirety.

### BACKGROUND OF THE INVENTION

Black tea is a type of tea that is more oxidized than yellow, white, and green teas. Black tea is generally stronger in flavour than other teas. All five types are made from leaves of Camellia sinensis, though Camellia taliensis may be also used.

Black tea contains 2 to 4 percent caffeine. The caffeine content of tea is affected by factors such as processing and brewing time. A standard cup of black tea (240 ml) contains 47 mg of caffeine.

The visible film often formed on black tea consists of oxidized polyphenols and calcium carbonate.

Black teas from Camellia sinensis contain flavonoids, which are under preliminary research for their potential to affect blood pressure and blood lipids as risk factors for cardiovascular disease. Long-term consumption of black tea only slightly lowered systolic and diastolic blood pressures (about 1-2 mmHg). Black tea consumption may be associated with a reduced risk of stroke, but there is only limited research to evaluate this possibility.

Green tea is a type of tea that is made from Camellia sinensis leaves and buds that have not undergone the same withering and oxidation process which is used to make oolong teas and black teas. Several varieties of green tea exist, which differ substantially based on the variety of C. sinensis used, growing conditions, horticultural methods, production processing, and time of harvest. Polyphenols found in green tea include epigallocatechin gallate (EGCG), epicatechin gallate, epicatechins and flavanols, which are under laboratory research for their potential effects in vivo. Other components include three kinds of flavonoids, known as kaempferol, quercetin, and myricetin. Although the mean content of flavonoids and catechins in a cup of green tea is higher than that in the same volume of other food and drink items that are traditionally considered to promote health, flavonoids and catechins have no proven biological effect in humans. Drinking green tea or taking green tea supplements decreases the blood concentration of total cholesterol (about 3-7 mg/dL), LDL cholesterol (about 2 mg/dL), and does not affect the concentration of HDL cholesterol or triglycerides. A 2013 Cochrane meta-analysis of longer-term randomized controlled trials (>3 months duration) concluded that green tea consumption lowers total and LDL cholesterol concentrations in the blood.

There is a need for a composition of tea and other agent that provides brain enhancement, awakening, that is long lasting. Further, there is a need to combine the cardiovascular effects of the tea with a plant cell culture composition grown in vitro that increases vascular function and dilation of arteries, improves blood flow, physical energy and mental alertness, or oxidation of blood lipids. In addition, it would be beneficial to combine tea with a plant cell culture composition grown in vitro that contain other active ingredients that are beneficial to the customers.

### SUMMARY OF THE INVENTION

In some embodiments, there is provided a tea composition comprising tea and a composition comprising plant cell culture grown in vitro, optionally in a form of a powder. In some embodiments, the combined tea composition is being added to a pod or container or bag.

In some embodiments, the combined tea composition provided in a pod is used in a machine.

In some embodiments, the resulted composition after tea beverage preparation comprises grape berry and tea polyphenols and piceid resveratrol.

In some embodiments, the resulted composition after tea beverage preparation comprises resveratrol in an amount of between 1-15 mg.

In some embodiments, wherein the smell and taste of the tea are maintained, i.e. the addition of the comprising plant cell culture grown in vitro does not change the taste and smell of the tea.

In some embodiments, there is provided a combined tea composition comprising a) tea and b) a composition comprising plant cell culture grown in vitro, optionally in a form of a powder.

In some embodiments, the tea is a green tea or a black tea.

In some embodiments, the combined tea composition is served in bag, a pod or container.

In some embodiments, the combined tea composition is used in a machine.

In some embodiments, plant cell culture grown in vitro is grape berry cell culture grown in vitro.

In some embodiments, the plant cell culture grown in vitro is olive cell culture grown in vitro.

In some embodiments, the plant cell culture grown in vitro is pomegranate cell culture grown in vitro. In some embodiments, the tea includes caffeine.

In some embodiments, the tea is decaffeinated.

In some embodiments, the weight ratio between the tea and the composition comprising plant cell culture grown in vitro is between about 20:1 to 1:3.

In some embodiments, the weight ratio between the tea and the composition comprising plant cell culture grown in vitro is between about 10:1 to 3:1.

In some embodiments, the weight ratio between the tea and the composition comprising plant cell culture grown in vitro is between about 8:1 to 3:1.

In some embodiments, the weight ratio between the tea and the composition comprising plant cell culture grown in vitro is between about 5:1 to 3:1.

In some embodiments, the resulted composition after extraction comprises grape berry polyphenols and piceid resveratrol.

In some embodiments, the particle size of the tea is between 200-900 μm.

In some embodiments, the particle size of the RGC culture powder grown in vitro is between 90-220 μm.

In some embodiments, the density of the RGC culture powder grown in vitro is 0.26-0.56 g/ml.

In some embodiments, the level of resveratrol in the RGC powder is 1% or more.

In some embodiments, the particle size of the plant cell culture is homogenous.

In some embodiments, the combined tea composition is cold brewed.

In some embodiments, the combined tea composition improves vascular function and dilation of arteries.

In some embodiments, the combined tea composition improves blood flow.

In some embodiments, the combined tea composition improves physical energy and mental alertness.

In some embodiments, the combined tea composition improves oxidation of blood lipids.

In some embodiments, the combined tea composition is antioxidant.

In some embodiments, the plant cell culture grown in vitro contains less than 10%, 8%, 6%, 4%, 2% or less sugar.

In some embodiments, the smell and taste of the tea are maintained.

### DETAILED DESCRIPTION OF THE EMBODIMENTS OF THE INVENTION

In some embodiments, the invention relates to a tea beverage composition containing tea and a composition comprising plant cell culture grown in vitro, which may be in a form of a powder. In some embodiments, the disclosure relates to a composition for use in a tea capsule or pod, which gives a good tasting and appearance of final beverage after the preparation step. In some embodiments, tea bags comprising tea and plant cell culture grown in vitro are provided.

The inventors have found that surprisingly, the mixing of a composition comprising plant cell culture grown in vitro with ground tea as described herein, results in a composition in which the tea taste is not different from a composition having tea only. Moreover, the beneficial effects of the composition comprising plant cell culture grown in vitro remain the same in spite of the extraction, which may be in high temperature and in a high pressure. As is detailed below the plant cell culture grown in vitro may be fruit cell culture grown in vitro. In some embodiments, the plant cell culture grown in vitro may be grape berry fruit, pomegranate or olive cell culture grown in vitro.

Surprisingly, the recovered level of piceid resveratrol was above 80% and the piceid resveratrol was stable despite the hot temperature of the water, for at least 5, 7, 10, 15, 20 or more minutes.

In some embodiments, the ground tea a particle size distribution of from about 200 to 900 μm; and the plant cell culture powder, in particular RGC has a particle size distribution of from 90 μm to 200 μm. In some embodiments, the density is from about 0.26 gr/ml to 0.56 g/ml.

In some embodiments, in order to ensure high recovery of the resveratrol piceid the level thereof in the in initial RGC powder should be 1% more. In some embodiments, in order to ensure high recovery of the piceid resveratrol, the RGC poeder should have a particle size distribution of from 90 μm to 200 μm. In some embodiments, the density is from about 0.26 gr/ml to 0.56 g/ml.

In some embodiments, the composition contains the tea and the composition of the plant cell grown in vitro in a ratio by weight of from 20:1 to 1:3. When the ratio falls outside of these ranges, the beneficial effects are not observed. Instead it is found that the tea is weak, either because of a lack of tea, or a poor extraction of the large amount of tea present, presumably due to its stickiness between the tea and the plant cell culture powder. In some embodiments the ratio by weight of the tea to the plant cell grown in vitro composition is from 10:1 to 3:1. In some embodiments the ratio by weight of the tea to the plant cell grown in vitro composition is from 5:1 to 3:1. According to some embodiments, the weight ratio between the tea and the composition comprising plant cell culture grown in vitro is between about 8:1 to 3:1. According to some embodiments, the weight ratio between the tea and the composition comprising plant cell culture grown in vitro is between about 24:1 to 20:1. According to some embodiments, the weight ratio between the tea and the composition comprising plant cell culture grown in vitro is between about 4:1. According to some embodiments, the weight ratio between the tea and the composition comprising plant cell culture grown in vitro is between about 5:1.

In some embodiments, the fruit cell culture grown in vitro is in vitro grown red grape cells (RGC or RGC product). RGC powder contains a matrix of polyphenols including piceid resveratrol, quercetin, anthocyanins, and tannins. In some embodiments, the RGC powder is composition in a form of a powder comprising a cell line culture of grape berry cells grown in vitro in a large scale up process, whereby the cell line culture of grape berry cells is derived from one or more of grape-berry cross section, grape-berry skin, grape-berry flesh, grape seed, grape embryo of seeded or seedless cultivars or grape seed coat; wherein the cell line culture of grape berry cells includes resveratrol in an amount of at least 1000 mg/kg powder. In some embodiments, the RGC is prepared as detailed in WO 2014/068557. In some embodiments, the RGC powder is equivalent to VINIA®. However, it is noted that the VINIA® product was not adequate for producing a composition of tea with RGC product. The inventors have found that in order to combine RGC with tea particles to a composition suitable for being inserted to a pod and used in a tea machine it is needed to produce RGC at an average particle size of about 60-150 μm. Also, the ratio between the tea and RGC product amounts is important. VINIA® is made of red grapes (Vitis vinifera) cells grown in vertical bioreactors and comes in a form of fine dry pink-purple powder containing the whole matrix of polyphenols with a high concentration of piceid resveratrol in their natural state that have additive and synergistic effects.

VINIA® is a complex of red grape cells from the red grape skin/flesh/pulp and seed. Accordingly, VINIA® is a source of the whole matrix of nutrients and polyphenols found throughout the red grape and in red wine in their natural state (physicochemical state) without any genetic manipulation and contains polyphenols (resveratrol, tannins, quercetin, catechins, anthocyanins), vitamins, protein, fiber. Its advantages are in the absence of solvent residuals
and the typical taste without bitterness and astringent.

In some embodiments, the plant cell culture grown in vitro is olive cell culture grown in vitro.

In some embodiments, the olive cells composition is a composition in a form of a powder comprising olive fruit/leaf cells grown in vitro grown in vitro in a large-scale process. Whereby the olive cells are derived from one or more of section: olive pulp, olive seed, olive petiole or olive leaf. wherein the olive fruit cells include polyphenols such as hydroxytyrosol, tyrosol, oleuropein, verbascoside and pinoresinol. The composition contains in one embodiment high amounts of polyphenols particularly, the secondary metabolites verbascoside. The unique composition of olive cells (OC), which, as an outcome of scale up process, includes a whole matrix of polyphenols and other healthy ingredients, naturally existing in OC of a plant, with higher concentration, i.e., at least 1%, 2%, 2.5%, 3%, 4%, 5%, 7%, 9%, 10%, 13%, 15%, 17%, 20% or higher verbascoside. As used herein the term “polyphenols” refers to naturally occurring phyto organic compounds having more than one phenol group. Polyphenols may range from simple molecules, such as phenolic acid, to large, highly polymerized, compounds such as hydrolyzed tannins. The phenolic rings of polyphenols are typically conjugated to various sugar molecules, organic acids and/or lipids. Differences in this conjugated chemical structure account for the chemical classification and variation in the modes of action and health properties of the various polyphenol compounds.

In some embodiments, the olive cell culture is manufactured as described in WO 2017/130199.

In some embodiments, the olive cell culture grown in vitro is a composition in a form of a powder comprising olive fruit/leaf cells grown in vitro grown in vitro in a large-scale process. Whereby the olive cells are derived from one or more of section: olive seed, olive fruit or olive leaf.

In some embodiments the Olive Cell Product is a dry cell line culture of olive cells manufactured in vitro in a large scale comprising verbascoside in an amount of 40-200 g/kg dry weight

In some embodiments the Olive Cell Product (OCP) is a dry cell line culture of olive cells manufactured in vitro in a large scale comprising total polyphenols in an amount of 50-220 g/kg dry weight, wherein the total polyphenols comprises verbascoside in an amount of 40-200 g/kg dry weight, 1-O-synapoylglucose and beta-hydroxyverbascoside and the product does not comprise detectable levels of tyrosol, hydroxytyrosol, and oleuropein.

In some embodiments, the OCP is manufactured in a large scale process for the in vitro production of an olive cell culture of olive cells grown comprising: growing olive cells in a flask;
inoculating the olive cells from the flask into a first bioreactor;
inoculating the olive cells from the first bioreactor into a second bioreactor;
optionally inoculating the olive cells from the second bioreactor into a last bioreactor; and
harvesting the olive cells from the last bioreactor;
wherein the second bioreactor is a last bioreactor or an intermediate bioreactor and wherein the olive cells harvested from the last bioreactor are dried.

According to some embodiments, the size of each bioreactor used in the process is larger than the one in which the olive cells were previously grown.

According to some embodiments, if the second bioreactor is an intermediate bioreactor, an additional step of inoculating the olive cells to another intermediate bioreactor or to the last bioreactor is performed.

According to some embodiments, the large scale process of the invention further includes additional steps of inoculating the olive cells from the second bioreactor into any number of sequential intermediate bioreactors.

In some embodiments, there is provided a process for the in vitro production of an olive cell culture of olive leaf, or olive fruit cells grown in vitro in a large scale comprising:
growing olive cells in a flask;
inoculating the olive cells from the flask into a first bioreactor;
inoculating the olive cells from the first bioreactor into a second bioreactor;
optionally inoculating the olive cells from the second bioreactor into a last bioreactor; and harvesting the olive cells from the last bioreactor;
wherein the second bioreactor is a last bioreactor or an intermediate bioreactor and the size of each bioreactor used in the process is larger than the one in which the olive cells were previously grown;
wherein the olive cells harvested from the last bioreactor are dried and wherein the olive cells are grown in bioreactors in a growth medium comprising between about 2-5% sucrose, 9-20 mg/l, ZnSO₄·7H₂O, 7-20 mg/l H₃BO₃, 0.1-1 mg/l, CuSO₄ 5H₂O, 400-900 mg/l MgSO₄, 100-500 mg/l Ca(NO₃)₂ mg/l and 150-500 mg/l KH₂PO₄, 350-650 mg/l KCl, 700-1800 mg/l KNO₃ or 200-1500 mg/l NH₄NO₃ between about 0.2-1 mg/l folic acid, between about 2-8 mg/l nicotinic acid, between about 0.2-1 mg/l thiamine, between about 0.02-0.08 mg/l biotin and pea pepton, wherein the olive fruit/leaf cells grown in vitro comprises verbascoside in the amount of at least between 40-200 g/kg dry weight and wherein the olive fruit/leaf cells grown in vitro do not comprise detectable levels of tyrosol, hydroxytyrosol, and oleuropein.

In some embodiments, if the second bioreactor is an intermediate bioreactor, an additional step of inoculating the olive cells to another intermediate bioreactor or to the last bioreactor is performed.

In some embodiments, the process further including additional steps of inoculating the olive cells from the second bioreactor into any number of sequential intermediate bioreactors.

In some embodiments, any one of the bioreactors is a 3-9 liter bioreactor.

In some embodiments, any one of the bioreactors is a 30-65 liter bioreactor. In some embodiments, any one of the bioreactors is a 30-200-liter bioreactor. In some embodiments, any one of the bioreactors is a 200-400 liter bioreactor. In some embodiments, any one of the bioreactors is a 200-1100 liter bioreactor. In some embodiments, any one of the bioreactors is a 1000-2000 liter bioreactor. In some embodiments, any one of the bioreactors is a 2000-5000 liter bioreactor. In some embodiments, any one of the bioreactors is a 2000-10000-liter bioreactor. In some embodiments, at least one bioreactor is a disposable bioreactor having airflow of more than 800 liter/h, 1000 liter/h, 1200 liter/h, 1500 liter/h or more.

In some embodiments, the disposable bioreactor is made from one or more layers of polyethylene. In some embodiments, the disposable bioreactor includes an inner and an outer layer prepared from polyethylene and a middle layer prepared from nylon. In some embodiments, the growth medium further comprises a pea pepton.

In some embodiments, the pea pepton is in amount of between 50 to 500 g/l. In some embodiments, the pea pepton is in amount of between 100 to 400 g/l. In some embodiments, wherein the pea pepton is in amount of between 200 to 300 g/l. In some embodiments, the growth medium is enriched with one or more of vitamin, cytokine, auxin, glycine, myo inositol, nicotinic acid, pyridoxine, biotin, thiamine, folic acid, 2,4-D, or 2 iP. In some embodiments, the amount of 2, 4-D is between 0.3-0.6 mg/l. In some embodiments, the amount of 2 iP is between 0.07-0.2 mg/l. In some embodiments, the amount of glycine is between 0.5-6 mg/l. In some embodiments, the amount of myo inositol is between 50-200 mg/l. In some embodiments, the amount of nicotinic acid is between 2-8 mg/l. In some embodiments, the amount of pyridoxine HCl is between 0.1-1 mg/l. In some embodiments, the amount of thiamine HCl is between 0.2-0.8 mg/l. In some embodiments, the amount of biotin is between 0.01-0.1 mg/l. In some embodiments, the amount of folic acid is between 0.2-0.8 mg/l.

In some embodiments, there is provided a process for the in vitro production of an olive cell culture of olive leaf, or olive fruit cells grown in vitro in a large scale comprising: growing olive cells in a flask; inoculating the olive cells from the flask into a first bioreactor; inoculating the olive cells from the first bioreactor into a second bioreactor; optionally inoculating the olive cells from the second bioreactor into a last bioreactor; and harvesting the olive cells from the last bioreactor; wherein the second bioreactor is a last bioreactor or an intermediate bioreactor and the size of each bioreactor used in the process is larger than the one in which the olive cells were previously grown; wherein the olive cells harvested from the last bioreactor are dried and wherein the olive cells are grown in bioreactors in a growth medium comprising CoCl₂·6H₂O, CuSO4, FeNaEDTA, H₃BO₃, KI, MnSO₄·H₂O, Na₂MoO₄·2H₂O, ZnSO₄·7H₂O, CaCl₂, Ca (NO_{3) 2}, KH₂PO₄, KNO₃, Ca(NO₃)₂, NH₄NO₃, MgSO₄ or KCl; Pea pepton; and one or more of vitamin, auxin, cytokine, glycine, sucrose, myo inositol, nicotinic acid, pyridoxine, biotin, thiamine and folic acid. In some embodiments, wherein the growth medium comprises:

| Medium | mg/l |
|---|---|
| CoCl₂•6H₂O | 0.01-0.04 |
| CuSO₄•5H₂O | 0.1-0.5 |
| FeNaEDTA | 20-53 |
| H₃BO₃ | 9-15 |
| Kl | 0.60-0.9 |
| MnSO₄•H₂O | 13-19 |
| Na₂MoO₄•2H₂O | 0.15-0.5 |
| ZnSO₄•7H₂O | 10.5-16 |
| CaCl₂ | 283-360 |
| KH₂PO₄ | 250-500 |
| KNO₃ | 900-1260 |
| MgSO₄ | 400-900 |
| NH₄NO₃ | 300-480 |
| Ca(NO₃)2 | 100-500 |
| KCl | 350-650 |

Pea pepton; and one or more of vitamin, cytokine, auxin s glycine, sucrose, myo inositol, nicotinic acid, pyridoxine, biotin, thiamine and folic acid.

In some embodiments, the amount of pea peptone and the one or more of vitamin, auxin, cytokine, glycine, sucrose, myo inositol, nicotinic acid, pyridoxine, biotin, thiamine and folic acid is as follows:

| | mg/l |
|---|---|
| Pea peptone | 100-500 |
| Sucrose | 2-5% |
| Glycine | 1.8-3.0 |
| myo-Inositol | 88-115 |
| Nicotinic acid | 4-7 |
| Pyridoxine HCl | 0.4-0.7 |
| Thiamine HCl | 0.4-0.8 |
| Biotin | 0.02-0.08 |
| Folic acid | 0.3-0.65 |

In some embodiments, the cells are grown in OCR medium at the stage of Erlenmeyers, wherein the medium is supplemented with pea pepton and sucrose. In some embodiments, the cells are grown in MS medium at the stage of Erlenmeyers, wherein the medium is supplemented with pea pepton and sucrose.

In some embodiments, the plant cell culture grown in vitro is pomegranate cell culture grown in vitro. The composition is in a form of a powder comprising a cell culture of pomegranate cell culture (PC) grown in vitro in, whereby the cell culture of PC is derived from one or more of pomegranate sections: pomegranate skin exocarp, mesocarp, pomegranate lamellae and pomegranate seeds. In an embodiment of the invention, the cell culture of PC includes punicalagin and PGG in an amount of at least 1300 mg punicalagin/kg powder and 2600 mg PGG/kg powder. Typical pomegranate polyphenols include but not limited to ellagitannins (e.g. punicalagin and punicalin), gallic and ellagic acids and 1,2,3,4,6-pentagalloyl glucose (PGG). The pomegranate fruit may be of a wild or cultivated variety.

In some embodiments, the pomegranate cell culture is manufactured as described in WO 2015/102003.

In an embodiment of the invention, the combined tea composition may include more than one plant cell culture grown in vitro. For example, without being limited, the tea may be combined with red grape berry cell culture grown in vitro and olive cell culture grown in vitro.

In some embodiments, the combined tea composition of the invention further comprises one or more flavorings, selected from the group consisting of cream, almond, amaretto, anise, apple, brandy, caramel, cider, cinnamon, cherry, chocolate, mint, cocoa, panna, milk, creme de menthe, French vanilla, grape, hazelnut, soluble coffee, Irish cream, lemon, macadamia nut, orange, peach, peppermint, pistachio, strawberry, vanilla, wintergreen or a mixture of two or more thereof. The one or more flavorings are present in an amount of less than 5 wt %, 4 wt %, 3 wt %, 2 wt % or less than 1 wt % of the composition.

In some embodiments, the combined tea composition of the invention does not contain sugar. In some embodiments, the plant cell culture grown in vitro that is added to the tea contains less than 10%, 8%, 6%, 4%, 2% or less sugar. According to another embodiment there is provided a container comprising the composition disclosed herein. The container is suitable for a beverage preparation machine. Such containers are well known in the art and include various pods, pads, sachet, bags, capsules and cartridges, which are optionally made of plastic, paper, polybutylene perephthalate (PBT), food grade plastic with aluminium foil lids and aluminium capsules.

According to a further aspect there is provided a kit comprising a plurality of the containers disclosed herein.

According to some embodiments, the combined tea composition includes caffein.

According to some embodiments, the combined tea composition includes a decaffeinated tea.

According to some embodiments, if the plant cell culture is grape berry cell culture, the resulted composition in the prepared tea beverage after extraction comprises resveratrol and/or piceid resveratrol.

According to some embodiments, if the plant cell culture is grape berry cell culture, the resulted composition after extraction in the prepared tea beverage comprises resveratrol and/or piceid resveratrol in an amount of between 1-15 mg.

According to some embodiments, if the plant cell culture is grape berry cell culture, the resulted composition after extraction comprises resveratrol in an amount of between 3-8 mg.

According to some embodiments, if the plant cell culture is grape berry cell culture, the resulted composition after extraction comprises resveratrol in an amount of between 5-7 mg.

According to some embodiments, if the plant cell culture is grape berry cell culture, the resulted composition after extraction comprises resveratrol in an amount of about 6 mg.

It is noted that the term “resveratrol” herein refers to all resveratrol forms such as: cis piceid resveratrol, trans piceid resveratrol, cis resveratrol and trans resveratrol.

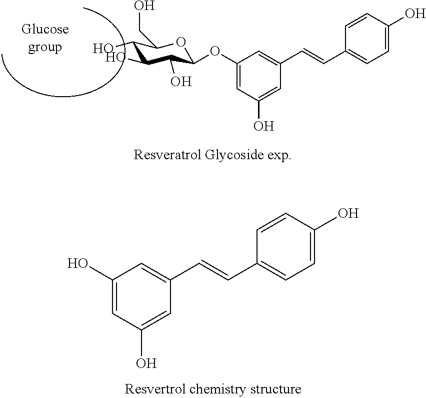

Chemistry structure of the RGC Product, (VINIA®)

RGC Product, (VINIA®) is Red Grape Cells a natural product consists of ingredients associated with Red Wine such as polyphenols, mainly piceid resveratrol, maintaining the quality and inherent benefits present in nature without any solvent extraction or genetic modification. RGC is a fine pink-purple powder of cells originating from fruit of red grape berry (Vitis Vinifera), which are grown in aqueous media consisting of water and defined nutrients in bioreactors under controlled conditions such as light and temperature. Red grape cells are grown in large scale bioreactors grape cells yield a product that has marked advantages over the current alternative commercial products since it whole complex of polyphenols of grape constituents and a significant higher concentration of grape resveratrol-glycoside (piceid) than in current grape derived products, in their natural state, that have additive and synergistic benefits.

RGC also contains in addition to piceid resveratrol polyphenols, tannins, quercetin, catechins, anthocyanins, vitamins (B1, B6, C), protein, fiber, minerals and low level of sugar. The Natural state of piceid resveratrol in RGC permits better absorption and long-lasting ability.

Many studies have shown that glycosides differ from the parent aglycones molecule (molecules without the sugar/glucose group), not only in their antioxidant and biological activities, but also in their water solubility and bioavailability (Goldberg et al., 1996; Spencer et al., 2001; Kuhnle et al., 2000; Orsini et al., 1997; Stojanovic et al., 2002; Stivala et al., 2001; Biasutto et al., 2009).

Resveratrol can be found in a plant as both a glycoside and aglycone. In the production of most resveratrol end-products, the sugar molecule is stripped from the glycoside resveratrol or it is produced as a synthetic molecule as an aglycone, without the glucose group.

Indeed, the glycoside form of trans-resveratrol, identified as piceid, is the predominant form of resveratrol found in VINIA® (99% piceid or glycoside resveratrol). The RGC production process is a controlled process favoring the production of piceid esveratrol while preventing its destruction in the cells. This enables a high and stable VINIA® piceid concentration. VINIA is fast absorbing. The glycosylated structure enables VINIA's resveratrol to be more soluble in aqueous media such as body fluids. Accordingly, VINIA's resveratrol presented higher water solubility compared to other sources of resveratrol (Azachi et al., 2014). The high water solubility of the glycosyl groups in VINIA's resveratrol explains VINIA's significantly high gastrointestinal absorption rate, as demonstrated in Azachi et al. 2014, when resveratrol was found in the plasma samples as soon as 20 minutes after ingestion.

Red grapes are an important source of resveratrol, one of the most investigated plant polyphenols with a myriad of beneficial health effects. In plants, polyphenols like resveratrol, are usually found as glycosides, sugar molecules bound to the polyphenols (Alluis et al., 2001; Goldberg et al., 1996).

The inventors have surprisingly found that the combined tea composition of the invention even after the step of heating and/or high pressure to produce tea beverage into a cup or bag maintains the metabolite structure, composition and function the RGC as described above.

According to some embodiments, the particle size of the tea is between 200-900. According to some embodiments, the particle size of the tea is between 200-700. According to some embodiments, the particle size of the tea is between 200-600. According to some embodiments, the particle size of the tea is between 200-500. According to some embodiments, the particle size of the tea is between 200-400. According to some embodiments, the particle size of the tea is between 300-700. According to some embodiments, the particle size of the tea is between 300-600.

According to some embodiments the plant cell culture grown in vitro, such as the RGC powder, is homogenous.

According to a further aspect there is provided a method of manufacturing a beverage in a beverage preparation machine, the method comprising: introducing the container described herein into a beverage preparation machine; and passing an aqueous medium through the container to produce a beverage.

The aqueous medium will typically be water, although another prepared beverage may also be used, such as from another container. The medium is heated during the process, and may be heated to a temperature of from 70 to 95° C.

The container is suitable for producing a beverage having a volume of 50 to 400 ml, preferably from 100-250 ml.

In a human clinical trial VINIA® was found to significantly increase vascular function and dilation of arteries as was demonstrated by significant increasing of FMD after consuming 400 mg daily over a 90-day period. This promotes the delivery of increased blood flow and oxygen to the body's tissue and organs supporting improved physical energy and mental alertness. Flow-mediated dilation (FMD) as an expression of endothelial dysfunction may serve as an indicator of several diseases.

Further, as shown in U.S. Pat. No. 9,867,861, vascular function was measured by FMD in six subjects that received 200 mg RGC, eight subjects that received 400 mg RGC, and in nine subjects who received a placebo. A statistically significant increase of 2.14±1.82 mm Hg in FMD was observed in the RGC 400 mg group between baseline and the end of treatment (Table 17, p=0.013, therein). FMD was performed by inflating a blood pressure cuff on the upper arm to 200 mm Hg for 5 minutes and then measuring the artery's dilation by ultrasonography after releasing the cuff.

All eight subjects (100%) from the 400 mg treatment group experienced a positive relative change (>70% in FMD) compared to two out of six subjects (33.3%) from the 200 mg treatment group and two out of nine subjects (22.2%) who received a placebo (FIG. 11). The improvement rate observed in the RGC 400 mg group was statistically significantly different from that observed for both the 200 mg group and the placebo group (p=0.015 for 400 mg .vs. 200 mg and p=0.0023 for 400 mg .vs. placebo).

The results presented in U.S. Pat. No. 9,867,861 indicate that a daily consumption of RGC powder improves FMD and also improves oxidative stress in mild hypertensive subjects. RGC may also decrease diastolic and systolic blood pressure in subjects with moderate hypertension who are not medically treated.

In some embodiments, the combined tea composition of the invention improves endothelial and vascular function and dilation of arteries.

In some embodiments, the combined tea composition of the invention the combined tea composition improves blood flow.

In some embodiments, the combined tea composition of the invention improves physical energy and mental alertness.

In some embodiments, the combined tea composition of the invention improves oxidation of blood lipids.

### EXAMPLES

Composition of tea with red grape cell powder (RGC) was developed to create a unique tea composition enriched in red grape constituents such as resveratrol and other polyphenols. The tea and RGC composition may be inserted into a pod and be used in a tea machine or inserted into a tea bag or a container. In all experiments, RGC powder was produced to create a fine purple powder with specific particle size of 90-200 um and bulk density of 0.26-0.56 g/ml.

### Example 1

Red grape cell culture grown in vitro powder (RGC) was added to different tea types as a food ingredient or dietary supplement to enhance the health benefits and nutritional profile of tea.

Different formulations of RGC with four types of tea: green tea, green tea matcha, black tea and cranberry tea were mixed with 450-500 mg RGC powder and the mixtures were inserted into tea bags. Tea beverages were prepared from these compositions and the level of piceid resveratrol recovery was measured in the beginning, middle, and end of a production batch using HPLC analysis.

### Experiment 1

### Composition of RGC and Black Tea

2.7-2.9 gram black tea was mixed with 500 mg RGC powder, in which the concentration of piceid resveratrol was 1.25% and the mixture was inserted into tea bags. Tea beverages were prepared from the RGC and black tea composition. The piceid resveratrol recovery was analyzed from the beginning, middle, and end of a production batch. The level of piceid resveratrol in the tea beverage was measured using HPLC analysis. Each tea bag was soaked in 177 mL hot water at 90-95° C. and the recovery and stability of piceid resveratrol in hot water was measured at different time points 1, 3 and 5 minutes were tested. Extraction yield was quantified by measuring the amount of piceid resveratrol obtained from each bag.

Recovered amounts of piceid resveratrol in tea bag were compared to the initial amounts exists in RGC powder.

### Results

TABLE 1
Recovery of piceid-resveratrol from black
tea with RGC bags at different time points

| | Tea Bag | | | |
|---|---|---|---|---|
| | Average | Incubation | RGC | Average |
| | Weight | Time | Weight | Recovery |
| Stage | (g) | (min) | (g) | (%) |
| Beginning | 2.9 | 5 | 500 | 86 |
| Middle | 2.7 | 1 | 500 | 91 |
| Middle | | 3 | 500 | 94 |
| Middle | | 5 | 500 | 93 |
| End | 2.7 | 5 | 500 | 94 |

### Conclusion

As can be seen in table 1, the composition of 2.7-2.9 g black tea and 500 mg RGC resulted in a high recovery of piceid resveratrol of 86-94% and the production was very homogenous as all stages of the production (beginning, middle and end). The piceid resveratrol from black tea with RGC was stable in the hot water for at least 5 minutes.

### Experiment 2

Compositions of RGC with Green Tea or with Matcha or Cranberry or with Green Tea with Matcha

Compositions of 460 mg RGC powder with 0.7% of piceid resveratrol were mixed with 2.9 g green tea with matcha or with 2.9 g cranberry tea and inserted into tea bags. Piceid resveratrol recovery was measured at different time points using HPLC analysis. Each tea bag was soaked in 177 mL hot water at 90-95° C. Recovered amounts of piceid resveratrol in tea bag were compared to the initial amounts initially exist in VINIA powder.

### Results

TABLE 2
Recovery of piceid-resveratrol from tea with
RGC bags at different and time points

| | RGC | Incubation | Average Recovery |
|---|---|---|---|
| | Weight | Time | Piceid |
| Tea type | (g) | (min) | resveratrol (%) |
| Green Tea with | 460 | 2 | 70 |
| Matcha-2.9 gr | | 3 | 65 |
| | | 5 | 60 |
| Cranberry Herbal | 460 | 1 | 62 |
| Tea 2.9 gr | | 5 | 60 |
| | | 7 | 57 |

### Conclusion

As can be seen in table 2 the composition of 2.9 gr green tea with matcha or 2.9 cranberry tea that were combined with 460 mg RGC where the level of resveratrol in the RGC was low 0.7% resulted in a low recovery of piceid resveratrol in average of 62%.

### Experiment 3

Formulations of 460 mg RGC in which the level of piceid resveratrol in RGC was 1.4% was combined with four types of tea (2.9 g of each of a. green tea; b. green tea with matcha; c. black tea and d. cranberry tea, which is decaffeinated) and inserted into tea bags. Tea beverages were prepared from these different compositions. The piceid resveratrol recovery was measured in all composition types from the beginning, middle, and end of a production batch. The level of piceid resveratrol in the tea beverage was measured using HPLC analysis.

TABLE 3
Recovery of Piceid-Resveratrol in different tea
compositions incubated for different times

| | | Average Recovery |
|---|---|---|
| | Incubation Time | Piceid |
| Tea type | (min) | resveratrol(%) |
| English Breakfast | 3 | 100 |
| Black tea- 2.9 gr | 5 | 98 |
| | 10 | 100 |
| Green Tea-2.9 gr | 3 | 100 |
| | 7 | 100 |
| | 10 | 100 |
| Green Tea with | 2 | 93 |
| Matcha-2.9 gr | 3 | 93 |
| Cranberry Herbal | 3 | 94 |
| Tea 2.9 gr | 5 | 99 |
| | 7 | 94 |

### Conclusion

As shown in Table 3 the four different tea compositions Black tea, Green Tea, Green Tea with Matcha and Cranberry Tea with RGC demonstrated high recovery of piceid resveratrol due to the specific particles size and its density of RGC powder and of level of piceid resveratrol in RGC that was above 0.7%. The average piceid recovery for all evaluated tea blends was determined to be between 93% and 100%. Surprisingly, the piceid resveratrol was stable in hot water for at least 10 minutes.

### Concluding Remarks:

These examples emphasize the significance of selecting specific parameters of RGC to be able to receive unique composition with high and stable recoveries of piceid resveratrol polyphenol. The parameters are for a. optimal bulk density of 0.26-0.56 gr/ml and particle size of 90-200 μm of the RGC powder which is essential. b. the level of piceid resveratrol in the initial RGC should be over 0.7%. The combination of these parameters is important to receive optimal and homogeneous piceid resveratrol recovery in hot tea beverages and further to obtain tea in which the levels of piceid resveratrol are stable in spite of the hot water.

### Example 2

Different formulations of Black and Green Tea with RGC in pods were prepared. 3.8 gr of green tea with matcha were mixed with 450 mg RGC powder and the mixture was inserted into pods. The level of piceid resveratrol in the initial RGC powder was 1.3%.

In addition, 4.6 gr black tea was mixed with 450 mg RGC, and the mixture was inserted into pods. The level of piceid resveratrol in RGC was 1.5%.

Tea beverages were prepared from these capsules using coffee machine (Keuring machine). The piceid resveratrol recovery was analyzed also from Black and Green Tea with matcha capsules sampled from the beginning, middle, and end of a production batch using HPLC analysis.

### Brewing and Measurement:

Small cup size, 160 ml
Medium cup size, 220 ml
Large cup size, 280 ml

### Results:

TABLE 4
Black Tea
% Recovery

| Small Cup | | Medium Cup | | Big Cup | |
|---|---|---|---|---|---|
| Beginning | 76.25 | Beginning | 96.71 | Beginning | 100 |
| Middle | 86.22 | Middle | 92.36 | Middle | 98.31 |
| End | 92.08 | End | 100 | End | 100 |
| Recovery | 85 | | 96 | | 99 |
| Average (%) | | | | | |

### Conclusion

As can be seen in table 4, the combination of 4.6 gr black tea and 450 mg RGC resulted in a good recovery of piceid resveratrol and the production was very homogenous as at different stages of the production has no significant effect on the % of recovered piceid resveratrol in the black tea drink. The recovery in the small cup is slightly lower which is not significant.

### Green Tea

Green tea

| | |
|---|---|
| Medium cup | Big cup |

| | | | |
|---|---|---|---|
| Beginning | 96.4 | Beginning | 93 |
| Middle | 83 | Middle | 94 |
| End | 88 | End | 83 |
| Average | 89 | | 90 |
| recovery (%) | | | |

### Conclusion

As can be seen in table 5, the combination of 3.8 gr green tea and 450 mg RGC resulted in a good recovery of piceid resveratrol and the production was very homogenous as the stage of the production has no significant effect on the recovery of piceid resveratrol in the green tea drink.

What is claimed is:

1. A combined tea composition comprising a) tea and b) a composition comprising plant cell culture grown in vitro, optionally in a form of a powder.

2. The combined tea composition of claim 1, wherein the tea is a green tea or a black tea.

3. The combined tea composition of claim 1, wherein the combined tea composition is served in bag, a pod or container.

4. The combined tea composition of claim 1, wherein the combined tea composition is used in a machine.

5. The combined tea composition of claim 1, wherein plant cell culture grown in vitro is grape berry cell culture grown in vitro.

6. The combined tea composition of claim 1, wherein the plant cell culture grown in vitro is olive cell culture grown in vitro.

7. The combined tea composition of claim 1, wherein the plant cell culture grown in vitro is pomegranate cell culture grown in vitro.

8. The combined tea composition of claim 1, wherein the tea includes caffeine.

9. The combined tea composition of claim 1 wherein the tea is decaffeinated.

10. The combined tea composition of claim 1, wherein the weight ratio between the tea and the composition comprising plant cell culture grown in vitro is between about 20:1 to 1:3.

11. The combined tea composition of claim 1, wherein the weight ratio between the tea and the composition comprising plant cell culture grown in vitro is between about 10:1 to 3:1.

12. The combined tea composition of claim 1, wherein the weight ratio between the tea and the composition comprising plant cell culture grown in vitro is between about 8:1 to 3:1.

13. The combined tea composition claim 1, wherein the weight ratio between the tea and the composition comprising plant cell culture grown in vitro is between about 5:1 to 2:1.

14. The combined tea composition claim 5, wherein the resulted composition after extraction comprises grape berry polyphenols and piceid resveratrol.

15. The combined tea composition of claim 14, wherein the resulted composition after extraction comprises resveratrol in an amount of between 1-15 mg.

16. The combined tea composition of claim 5, wherein the particle size of the tea is between 200-900 μm.

17. The combined tea composition of claim 5, wherein the particle size of the RGC culture powder grown in vitro is between 90-220 μm.

18. The combined tea composition of claim 5, wherein the density of the RGC culture powder grown in vitro is 0.26-0.56 g/ml.

19. The combined tea composition of claim 5, wherein the level of resveratrol in the RGC powder is 1% or more.
